(19) United States
(12) Patent Application Publication
(10) Pub. No.: US 2007/0041498 A1
(43) Pub. Date: Feb. 22, 2007

(54) SYSTEM AND METHOD OF REMOTELY DIRECTING RADIATION THERAPY TREATMENT

(76) Inventors: Gustavo H. Olivera, Madison, WI (US); Thomas R. Mackie, Verona, WI (US); Kenneth J. Ruchala, Madison, WI (US); John H. Hughes, Madison, WI (US); Paul J. Reckwerdt, Madison, WI (US); Robert L. Cravens, Verona, WI (US); Jeffrey M. Kapatoes, Madison, WI (US); Eric Schnarr, McFarland, WI (US); Eric Schloesser, Mount Horeb, WI (US); Weiguo Lu, Madison, WI (US); David Murray, Tauranga (NZ)

Correspondence Address: MICHAEL BEST & FRIEDRICH, LLP, 100 E WISCONSIN AVENUE, MILWAUKEE, WI, 53202, US

(21) Appl. No.: 11/459,113

(22) Filed: Jul. 21, 2006

Related U.S. Application Data
(60) Provisional application No. 60/701,544, filed on Jul. 22, 2005.

(51) Int. Cl. A61N 5/10

(52) U.S. Cl. 378/65

(57) ABSTRACT

Systems and methods for utilizing remote access to a radiation therapy treatment system. Remote access to the radiation therapy treatment system can assist in providing quality assurance processes, service and maintenance procedures, patient monitoring, and statistical analysis.

### RELATED APPLICATIONS

This application claims the benefit of U.S. Provisional Patent Application No. 60/701,544; filed on Jul. 22, 2005; entitled SYSTEMS AND METHODS OF REMOTELY ACCESSING A RADIATION THERAPY TREATMENT SYSTEM; the entire content of which is incorporated herein by reference.

### BACKGROUND

Over the past decades, improvements in computers and networking, radiation therapy treatment planning software, and medical imaging modalities have been incorporated into radiation therapy practice.

### SUMMARY

There are many clinical processes, both for patient treatment and system quality assurance/maintenance that would benefit from remote technologies. The benefits of providing remote access to a radiation therapy treatment system are numerous. Remote access to a radiation therapy treatment system can address issues such as quality assurance, service and maintenance procedures, patient monitoring, and statistical analysis. Medical personnel and technicians can remotely access a radiation therapy treatment system allowing for a flexible system with various operational options. Medical personnel can analyze patient information and technicians can handle set-up and service issues from remote terminals.

In one embodiment, the invention provides a method of remotely reviewing a radiation therapy treatment plan for a patient. The method includes the acts of accessing a radiation therapy treatment system with a computer remote from the radiation therapy treatment system, monitoring a parameter of the radiation therapy treatment system, analyzing the parameter, comparing the parameter to a predefined range, and notifying personnel local to the radiation therapy treatment system if the parameter is not within the predefined range.

In another embodiment, the invention provides a method of treating a patient with radiation therapy. The method includes the acts of acquiring an image of a patient at a first location, generating a radiation therapy treatment plan for the patient, notifying medical personnel at a second location that a treatment plan has been generated for the patient, remotely accessing a computer that generated the treatment plan to review the treatment plan, and performing the treatment plan.

In another embodiment, the invention provides a method of remotely directing radiation therapy treatment of a patient. The radiation therapy treatment is delivered by a radiation therapy system. The method comprises the acts of remotely accessing patient information using a remote computer distinct from the radiation therapy system, processing at least a portion of the patient information, and providing at least a portion of the processed information to an on-site person at the radiation therapy system.

In another embodiment, the invention provides a method of remotely directing radiation therapy treatment of a patient. The radiation therapy treatment is delivered by a radiation therapy system. The method comprises the acts of remotely accessing patient information using a remote computer distinct from the radiation therapy system, processing at least a portion of the patient information, generating radiation therapy information (e.g., radiation therapy treatment plans, contours, patient prescriptions, and the like) based at least in part on the processed information, and providing the radiation therapy information to an on-site person at the radiation therapy system.

In another embodiment, the invention provides a system for remotely directing radiation therapy treatment of a patient. The radiation therapy treatment is delivered by a radiation therapy system. The system comprises a computer processor distinct from the radiation therapy system, and a software program stored in a computer readable medium distinct from the radiation therapy system and accessible by the computer processor. The software program is executable by the computer processor to access patient information, process at least a portion of the patient information, and provide at least a portion of the processed information to an on-site person at the radiation therapy system.

Other aspects of the invention will become apparent by consideration of the detailed description and accompanying drawings.

### BRIEF DESCRIPTION OF THE DRAWINGS

FIG. 1 is a partial perspective view, partial schematic illustration of a radiation therapy system.

FIG. 2 is a schematic illustration of the radiation therapy system of FIG. 1.

FIG. 3 is a schematic illustration of a network for remote access to the radiation therapy system of FIG. 1

FIG. 4 is block diagram of a software program that can be used in the radiation therapy system of FIG. 1 or a remote computer of FIG. 3.

FIG. 5 is a block diagram of a software program that can be used in the remote computer of FIG. 3.

FIG. 6 is a flow chart illustrating a method of operation of the software programs of FIGS. 4 and 5 according to one embodiment of the invention.

FIG. 7 is a flow chart illustrating a method of operation of the software programs of FIGS. 4 and 5 according to one embodiment of the invention.

FIG. 8 is a flow chart illustrating a method of operation of the software programs of FIGS. 4 and 5 according to one embodiment of the invention.

FIG. 9 is a flow chart illustrating a method of operation of the software programs of FIGS. 4 and 5 according to one embodiment of the invention.

FIG. 10 is a flow chart illustrating a method of operation of the software programs of FIGS. 4 and 5 according to one embodiment of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the invention in any form. In addition, terms such as “first”, “second”, and “third” are used herein for purposes of description and are not intended to indicate or imply relative importance or significance.

In addition, it should be understood that embodiments of the invention include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

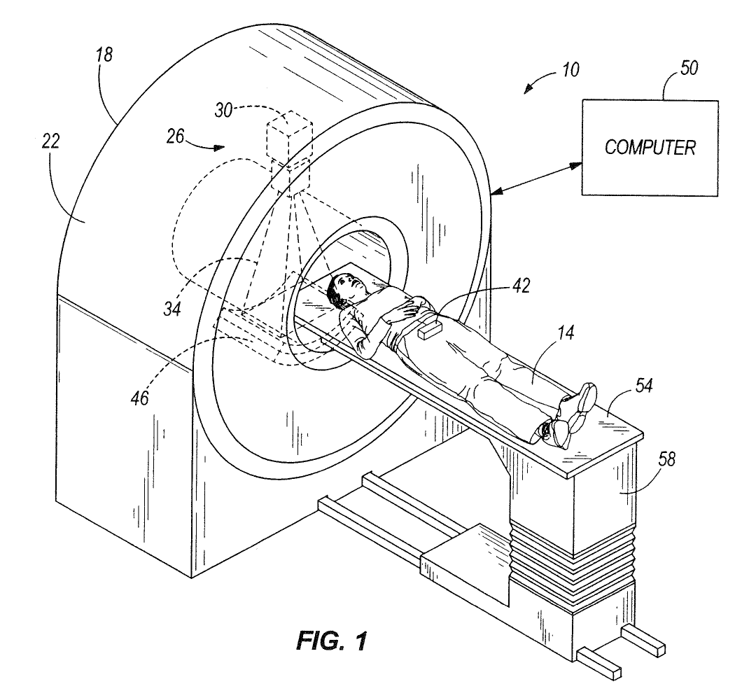

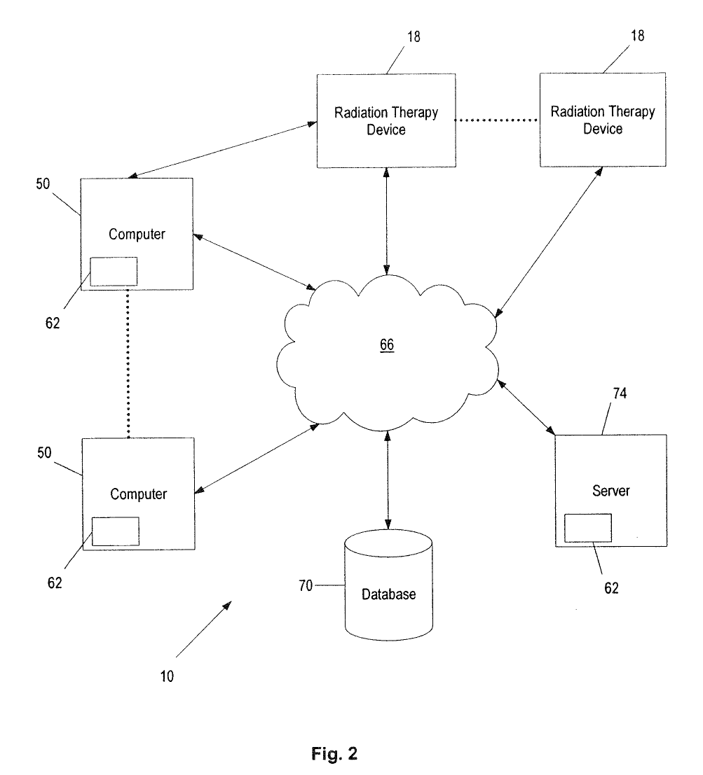

FIGS. 1 and 2 illustrate one construction of a radiation therapy system 10 that can provide radiation therapy to a patient 14. The radiation therapy treatment can include photon-based radiation therapy, brachytherapy, electron beam therapy, proton, neutron, or particle therapy, or other types of treatment therapy. The radiation therapy system 10 includes a radiation therapy device 18 having a gantry 22. Though the gantry 22 shown in the drawings is a ring gantry, i.e., it extends through a full 360° arc to create a complete ring or circle, other types of mounting arrangements may also be employed. For example, a C-type, partial ring gantry, or robotic arm could be used.

The gantry 22 can support a radiation module 26, having a radiation source and a linear accelerator (collectively shown as 30) operable to generate a beam 34 of photon radiation. The radiation module 26 can also include a modulation device 38 operable to modify or modulate the radiation beam 34. The modulation device 38 provides the modulation of the radiation beam 34 and directs the radiation beam 34 toward the patient 14. Specifically, the radiation beam 30 is directed toward a portion of the patient. Broadly speaking, the portion may include the entire body, but is generally smaller than the entire body and can be defined by a two-dimensional area and/or a three-dimensional volume. A portion desired to receive the radiation, which may be referred to as a target or target region (shown as 42), is an example of a region of interest. Another type of region of interest is a region at risk. If a portion includes a region at risk, the radiation beam is preferably diverted from the region at risk. The patient 14 may have more than one target region 42 that needs to receive radiation therapy. Such modulation is sometimes referred to as intensity modulated radiation therapy (“IMRT”).

Other frameworks capable of positioning the radiation module at various rotational and/or axial positions relative to the patient 14 may also be employed. In addition, the radiation module 26 may travel in path that does not follow the shape of the gantry 22. For example, the radiation may travel in a non-circular path even though the illustrated gantry 2 is generally circular-shaped.

The radiation therapy device 18 can also include a detector 46, e.g., a kilovoltage or a megavoltage detector, operable to receive a radiation beam from the treatment radiation source or from a separate radiation source. The linear accelerator and the detector 46 can also operate as a computed tomography (CT) system to generate CT images of the patient 14.

The CT images can be acquired with a radiation beam 34 that has a fan-shaped geometry, a multi-slice geometry or a cone-beam geometry. In addition, the CT images can be acquired with the linear accelerator 30 delivering megavoltage energies or kilovoltage energies.

The radiation therapy treatment system 10 can also include a patient support, such as a couch 54 (illustrated in FIG. 1), which supports the patient 14. The couch 54 moves along at least one axis in the x, y, or z directions. In other constructions, the patient support can be a device that is adapted to support any portion of the patient's body, and is not limited to having to support the entire patient's body. The system 10 also can include a drive system 58 operable to manipulate the position of the couch 54. The drive system 58 can be controlled by the computer 50.

As used herein, the term “computer” is broadly construed as an electronic device that receives, processes, and/or transmits information according to instructions. As used herein, the term “information” is broadly construed to include signals or data. The computer 50, illustrated in FIG. 2, includes an operating system for running various software programs and/or communication applications. In particular, the computer 50 can include a software program 62 operable to communicate with the radiation therapy device 18. The computer 50 can include any suitable input/output device adapted to be accessed by medical personnel. The computer 50 can include typical hardware such as a processor, I/O interfaces, and storage devices or memory. The computer 50 can also include input devices such as a keyboard and a mouse. The computer 50 can further include standard output devices, such as a monitor. In addition, the computer 50 can include peripherals, such as a printer and a scanner.

The radiation therapy device 18 communicates directly with the computer 50 and/or via a network 66 as illustrated in FIG. 2. The radiation therapy device 18 also can communicate with other radiation therapy devices 18 via the network 66. Likewise, the computer 50 of each radiation therapy device 18 can communicate with a computer 50 of another radiation therapy device 18. The computers 50 and radiation therapy devices 18 can also communicate with a database 70 and a server 74. A plurality of databases 70 and servers 74 can also communicate with the network 66. It is noted that the software program 62 could also reside on the server 74.

The network 66 can be built according to any networking technology or topology or combinations of technologies and topologies and can include multiple sub-networks. Connections between the computers 50 and devices 18 shown in FIG. 2 can be made through local area networks (“LANs”), wireless area networks (“WLANs”), wide area networks (“WANs”), public switched telephone networks (“PSTNs”), Intranets, the Internet, or any other suitable networks. In a hospital or medical care facility (collectively referred to as a health-care facility), communication between the computers 50 and devices 18 shown in FIG. 2 can be made through the Health Level Seven (“HL7”) protocol with any version and/or other required protocol. HL7 is a standard protocol that specifies the implementation of interfaces between two computer applications (sender and receiver) from different vendors for electronic data exchange in health care environments. HL7 can allow health care institutions to exchange key sets of data from different application systems. Specifically, HL7 can define the data to be exchanged, the timing of the interchange, and the communication of errors to the application. The formats are generally generic in nature and can be configured to meet the needs of the applications involved.

Communication between the computers 50 and radiation therapy devices 18 shown in FIG. 2 can also occur through the Digital Imaging and Communications in Medicine (DICOM) protocol with any version and/or other required protocol. DICOM is an international communications standard developed by the National Electrical Manufacturers Association (NEMA) that defines the format used to transfer medical image-related data between different pieces of medical equipment. DICOM RT refers to the standards that are specific to radiation therapy data.

The two-way arrows in the drawings generally represent two-way communication and information transfer between the network 66 and any one of the computers 50, the radiation therapy devices 18, and other components shown in the drawings. However, for some medical equipment, only one-way communication and information transfer may be necessary.

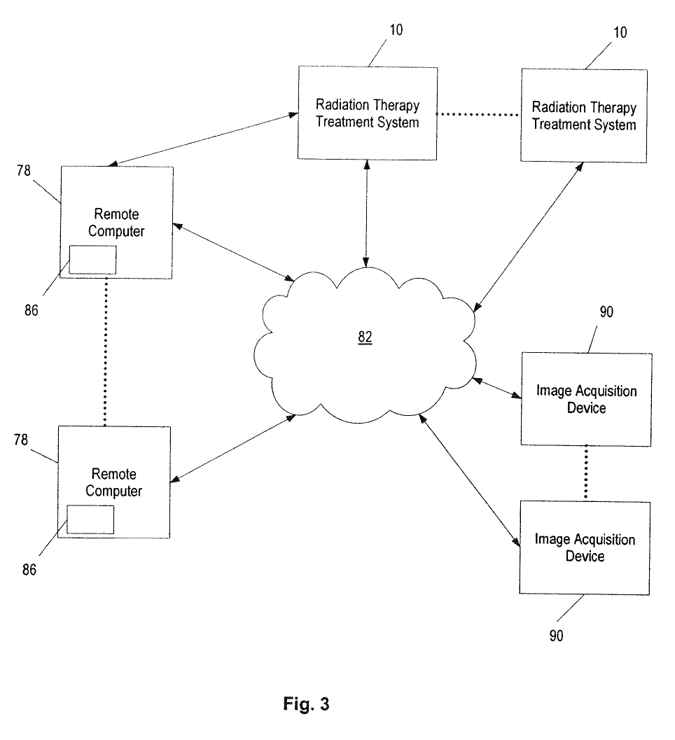

FIG. 3 schematically illustrates a radiation therapy system 10 that can be accessed by a remote computer 78 via a network 82. The remote computer 78 can be a handheld device, such as a PDA or tablet PC. The remote computer 78 can access the radiation therapy system 10, which is distinct from the remote computer 78. Before proceeding further, it should be understood that the remote computer 78 may or may not be located in the same facility as the radiation therapy system 10 (or the image acquisition device 90), and the computer 50 may or may not be located in the same room as the radiation therapy device 18. It is conceivable, for example, that the computer 50 not be proximate to the radiation therapy device 18, the remote computer 78 to be located in the same facility as the radiation therapy system 10, but that the remote computer 78 be distinct from the radiation therapy system 10 (including the computer 50).

The remote computer 78 includes an operating system for running various software programs and/or communication applications. In particular, the remote computer 78 can include a software program 86 operable to communicate with the radiation therapy system 10, the network 82, and other software for remote applications and communications. The remote computer 78 can include any suitable input/output device adapted to be accessed by medical personnel. The remote computer 78 can include hardware such as a processor, I/O interfaces, and storage devices or memory. The remote computer 78 can also include input devices such as a keyboard and a mouse, touch screen monitor. The remote computer 78 can further include standard output devices, such as a monitor. In addition, the remote computer 78 can include peripherals, such as a printer and/or a scanner.

The remote computer 78 enables medical personnel and technicians access to the radiation therapy system 10 while being on the move or in process of changing locations. As one example, medical personnel can view patient treatment history as well as edit and approve patient treatment plans without being at the site of the radiation therapy system 10. Medical personnel also can generate, view, and edit contours, which are generated to identify the regions of interest in the CT images of the patient 14 and the target 42. The contours also define the boundaries and the amount of radiation that a specific area or space of the target 42 will receive. Medical personnel also can approve or modify the treatment plan for a patient while at a remote location. The remote computer 78 provides a tool for medical personnel to manage patient and treatment information while providing mobility and convenience to the medical personnel.

The network 82 can be built according to any networking technology or topology or combinations of technologies and topologies and can include multiple sub-networks. Connections between the remote computers 78 and radiation therapy systems 10 shown in FIG. 3 can be made through local area networks (“LANs”), wireless area networks (“WLANs”), wide area networks (“WANs”), public switched telephone networks (“PSTNs”), intranets, the Internet, or any other suitable networks. In a hospital or medical care facility, communication between the remote computers 78 and radiation therapy systems 10 shown in FIG. 3 can be made through the Health Level Seven (“HL7”) protocol with any version and/or other required protocol. HL7 is a standard protocol that specifies the implementation of interfaces between two computer applications (sender and receiver) from different vendors for electronic data exchange in health care environments. HL7 can allow health care institutions to exchange key sets of data from different application systems. Specifically, HL7 can define the data to be exchanged, the timing of the interchange, and the communication of errors to the application. The formats are generally generic in nature and can be configured to meet the needs of the applications involved.

Communication between the remote computers 78 and the radiation therapy systems 10 shown in FIG. 3 can also occur through the Digital Imaging and Communications in Medicine (DICOM) protocol with any version and/or other required protocol. DICOM is an international communications standard developed by NEMA that defines the format used to transfer medical image-related data between different pieces of medical equipment. DICOM RT refers to the standards that are specific to radiation therapy data.

Communication can also occur through remote access to the computer interface and/or through a web-type interface (e.g., java, html, etc.) Communication can also occur through images of the relevant data such as a screen image of a plan viewed over the web without having to actually commandeer the planning computer.

The radiation therapy system 10 can communicate with and import and export data from one or more image acquisition devices 90, as illustrated in FIG. 3. In addition, the remote computers 78 can communicate with the image acquisition device 90.

The two-way arrows in FIG. 3 generally represent two-way communication and information transfer between the network 82 and any one of the remote computers 78, the radiation therapy systems 10, and other components shown in FIG. 3. However, for some medical equipment, only one-way communication and information transfer may be necessary. It should also be understood that the communication of information can be via a transmission or delivery of information and/or can be via making the information available (e.g., at a web site) for acquisition.

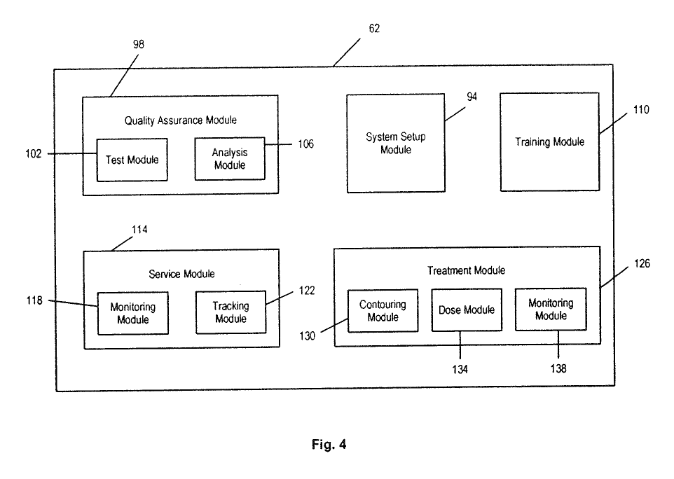

One exemplary software program 62 is schematically illustrated in FIG. 4. The software program 62 can be accessed remotely by the remote computer 78 and software program 86. The remote computer 78 communicates with the network 82 and the radiation therapy system 10 (computer 50 and/or radiation therapy device 18).

It is noted that various components and modules are discussed below with respect to the software program 62, however some or all of the components and modules could also be implemented in the software program 86. It is also noted that the processing activities could occur at either the computer 50, remote computer 78, and/or server 74. One particular benefit of remote processing of data is the opportunity for improved speed.

The software program 62 includes a system setup module 94 operable to configure the radiation therapy device 18. The system setup module 94 is also operable to determine whether the device 18 is properly commissioned, that the output and geometry of the modulation device 38 and imaging system are correctly modeled and within predetermined tolerances, and that the device 18 is ready for patient use. The system setup module 94 can also conduct predefined commissioning steps of the device 18, such as measurements of output, alignment, profiles, stability, geometry, couch performance, modulation device motion, gantry positioning/motion, and other device parameters.

The software program 62 also includes a quality assurance module 98 operable to conduct various tests and analyze the status and performance of the device 18. The quality assurance module 98 includes a test module 102 operable to conduct various tests on the device 18, such as radiation measurements, to verify proper operation. The personnel local to the radiation therapy device 18 (also referred to as the on-site personnel) can inform the remote site when to conduct tests and the types of tests to be conducted. Some of the tests may require that local personnel or a physicist perform a set of predefined preparatory steps, such as setting up jigs and phantoms, placing films, ion chambers, or other radiation measurement devices. These preparatory steps can be done by the local personnel before leaving for the evening or at other times when the device 18 is not being used. Other tests may require some local assistance, such as developing films or modifying setups.

The test module 102 is also operable to acquire and save data that is generated by performance of the tests. The test module 102 can retrieve patient specific data, such as data related to the delivery of a patient's treatment plan or future patient treatment plans, stored in the device 18 and/or computer 50.

The quality assurance module 98 also includes an analysis module 106 operable to analyze the data acquired from the tests that were conducted by the test module 102 and the patient specific data. The analysis module 106 evaluates the test results to determine if the device 18 is within predefined tolerances and otherwise in proper operational condition. The analysis module 106 can compare the test results of the device 18 to previous test results from the same device 18 and/or to test results from other radiation therapy devices 18. The analysis module 106 can evaluate delivery parameters of a treatment plan to determine if the device 18 delivered the treatment plan as expected. The analysis module 106 can also compare delivery parameters of more than one treatment plan of the same patient or different patients to determine if the device 18 delivered the treatment plan as expected. In some instances, the data results may help identify if the device 18 needs tuning and/or maintenance. The analysis module 106 can evaluate future patient treatment plans to verify that the plan and its associated device setup is suitable for delivery.

The analysis module 106 can specify whether local personnel need to take remedial action and/or identify whether additional tests or calibration should be performed on the device 18 if the analysis module 106 identifies an anomaly with the device 18 based on the test results. The analysis module 106 can also recommend changes to future patient treatment plans to compensate for changes that may be made to the device 18 as a result of retuning and/or maintenance.

The system setup module 94 and the quality assurance module 98 can improve the physics and quality assurance processes by offering consistency, automation, and efficiency. The features provided by the system setup module 94 and the quality assurance module 98 can be implemented in medical clinics (or elsewhere) that wish to save time in conducting the quality assurance processes for the device 18. The features offered by the modules 94 and 98 allow a medical clinic to receive oversight and training when beginning to use the device 18.

Medical personnel, at the remote computer 78, can instruct the test module 102 to perform a specified test of the device 18. The medical personnel, again from the remote computer 78, can instruct the analysis module 106 to evaluate the test results. Alternatively, the analysis module 106 can automatically analyze the test results. The analysis module 106 can transmit a report of the analysis results and/or recommendations to the remote computer 78 for review by the medical personnel.

The software program 62 also includes a training module 110 operable to monitor operation of the device 18 as medical personnel learn to operate and interact with the device 18. The training module 110 can provide step-by-step instructions for setup of the device 18 for quality assurance tests and/or for patient use. For example, the remote computer 78 can instruct the training module 110 to operate the device 18 and conduct various tests and/or operate according to a treatment plan while the medical personnel observes. Also, the training module 110 allows personnel at the remote computer 78 to monitor medical personnel as they operate the device 18. Personnel at the remote computer 78 can provide suggestions and advice to the local personnel on how to operate the device 18. Similarly, personnel at the remote computer 78 can monitor or supervise the local personnel during patient treatments. Training of medical personnel can be performed through the network 82 using the remote computer 78 to operate the training module 110 and radiation therapy device 18, and deliver instructions to trainees in real-time.

The software program 62 also includes a service module 114 operable to monitor component performance and reliability and environmental factors of the radiation therapy device 18. The service module 114 includes a monitoring module 118 operable to monitor environmental factors such as temperature, humidity, and air pressure of the room in which the device 18 is located. The monitoring module 118 is also operable to monitor parameters of the device 18, such as water flow, internal temperature, internal pressure, and the like. The monitoring module 118 can also monitor performance of external components, such as ion chambers, water tanks, diodes, film/film processors and the like. The monitoring module 118 can monitor in real-time the environmental factors, the device parameters, and the external components as the device 18 is in operation.

The service module 114 also includes a tracking module 122 operable to record and track the parameter data of the monitoring module 118. The tracking module 122 can compare the monitored parameter data to historical parameter data to identify device component problems. For example, the tracking module can compare recent parameter data relating to the beam of radiation from the radiation module 30 with historical parameter data relating to the beam of radiation from the radiation module 30. The tracking module 122 can automatically generate a report when a device component problem is identified and transmit the report to the remote computer 78. The tracking module 122 can generate a notification via phone, electronic mail, beeper, system messaging, or other modes of communication based on the type of component problem identified. In addition, the remote computer 78 can access the tracking module 122 to review the status of the parameter data to identify risk factors that indicate unsafe treatments to reduced machine stability to component failure. The remote computer 78 can instruct the service module 114 to correct the identified problem. For example, the remote computer 78 can instruct the service module 114 to retune or realign the device 18, change the room temperature, and schedule a component replacement.

The software program 62 also includes a treatment module 126 operable to perform functions related to patient treatment plans. There are numerous stages of the radiation therapy treatment process in which a clinical decision (or revision), approval, or judgment is necessary (collectively referred to as a decision point). Medical personnel interact with the treatment module 126 via the remote computer 78 to oversee multiple patients 14, treatment plans, and/or devices 18.

The treatment module 126 is operable to receive instructions from the remote computer 78, which allows medical personnel to view, edit, and/or approve patient plan optimization; view, edit, and/or approve patient contours; view, edit, and/or approve patient registration, and registration histories for a patient 14; view, edit, and/or approve adaptive therapy; view, edit, and/or approve quality assurance functions; view device history; view user history; view patient history; contact service/schedule maintenance; view data for other devices 18 or clinics; and transfer and/or triage patients to other devices 18 or clinics.

The treatment module 126 can include a contouring module 130 operable to generate contours on an image, such as a planning image. The contouring process is time consuming and may be outsourced to a remote center or to an automated system. The remote computer 78 can receive notification from the treatment module 126 that a treatment plan is waiting for the contours to be identified. The contouring task can be performed by trained and qualified personnel at the remote center. The local medical personnel can then approve, edit, or reject the remotely performed work, which in many cases could be done more efficiently. Alternatively, medical personnel can access the contouring module 130, via the remote computer 78, to view, edit, and/or approve the contours of a patient treatment plan.

The treatment module 126 also includes a dose module 134 operable to acquire patient radiation dose information after a treatment plan is delivered. The dose module 134 is operable to recalculate dose and/or perform deformation after each fraction based upon recent patient images, treatment parameters, and treatment feedback information, such as exit dose. The dose module 134 can process and analyze the dose data in accordance with specified tolerances. The dose module 134 can automatically transmit the data and analyzed results to the remote computer 78 for review. Medical personnel can review the dose data at the remote computer 78 and transmit suggestions back to the dose module 134 to make adjustments or determine whether the treatment is progressing according to the plan. The local personnel can review the suggestions made by the remote personnel and approve, alter, or reject the suggestions. The suggestions of the remote personnel could automatically be implemented if the local personnel provide a pre-approval for all suggestions, a sub-set of the suggestions, or changes that would fall within a predefined range made by the remote personnel.

The treatment module 126 also includes a monitoring module 138 operable to monitor all aspects of a treatment. The monitoring module 138 can include the use of video cameras that monitor the patient 14 and local medical personnel and windows into the device 18 and computer 50 that operate the device 18. The remote computer 78 can access the monitoring module 138 to monitor all aspects of radiation treatment from a remote location. The monitoring module 138 can be used for training, additional safety, or more efficiency. The remote computer 78 can access the monitoring module 138 such that remote medical personnel can view and/or adjust a treatment (e.g., positional parameters for gating, ultra sound, implantable markers, camera based tracking, detector data, and spirometric data) either in real-time or post-treatment. The monitoring module 138 can receive instructions from the remote computer 78 to adjust/discontinue treatment if certain tolerances are exceeded and/or predetermined protocols are not followed. The monitoring module 138 can generate a report or a notification to the remote computer 78 if certain tolerances are exceeded during treatment, or to indicate that treatment or a phase of treatment has been completed. Personnel at the remote computer 78 can notify other specified parties by phone, paging, electronic mail, or other modes of communication. Alternatively, the monitoring module 138 can notify other specified parties by phone, paging, electronic mail, or other modes of communication.

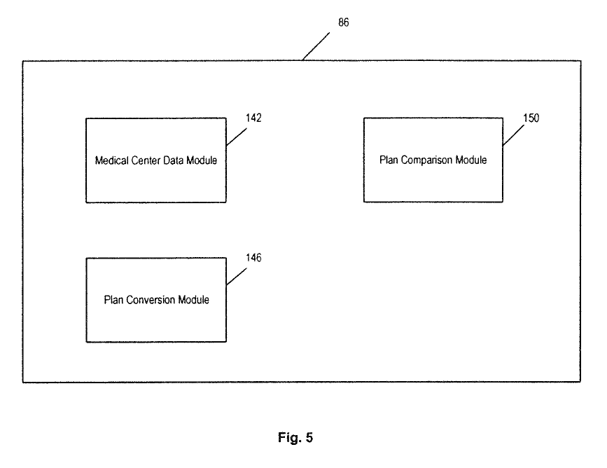

The software program 86 is schematically illustrated in FIG. 5. The software program 86 includes a medical center data module 142 operable to acquire and analyze throughput from a plurality of medical centers having radiation therapy systems 10. The medical center data module 142 can communicate with the computer 50 and the radiation therapy device 18 to retrieve data. The medical center data module 142 can organize and evaluate clinical throughputs on both a macroscopic (# of patients per day, etc.) level and a microscopic (speeds and delays related to particular steps of the treatment process) level. The medical center data module 142 can compare speeds for particular clinicians, treatment types, medical centers, etc. The medical center data module 142 can present options for improving medical center efficiency. For example, the medical center data module 142 could identify ways in which the slower medical centers or persons could improve, while also indicating how the radiation therapy system 10 could be improved based upon use. It could also allow for comparison of treatment plans, delivery times, opportunities for combined therapy, and outcomes with other centers.

The medical center data module 142 could facilitate scheduling for one or more medical centers by evaluating the speeds and workloads of the centers, along with the current patient load, machine downtime, patient distances to the different medical centers, and other information. Less tangible factors, such as patient willingness/unwillingness to travel, preference for particular clinical personnel, or interest in faster/slower fractionation schedules could also be incorporated. This queuing of patients could be performed for a single medical center or for a plurality of medical centers. Additional functionality can include the conversion of plans for running on different radiation therapy treatment devices 18, automated QA and physics necessary to run plans at different medical centers, remote adaptive therapy to monitor deliveries, accumulate dose, and adjust plans as needed, notification of relevant personnel, and remote consultation with primary clinicians.

The software program 86 also includes a plan conversion module 146 operable to convert treatment plans generated by different radiation therapy system manufacturers. The plan conversion module 146 can also convert treatment plans generated by radiation therapy systems 10 at different medical centers. The plan conversion module 146 analyzes the treatment plan and system settings generated by a radiation therapy system 10 of a first manufacturer to generate a treatment plan and system settings for a radiation therapy system 10 of a second manufacturer. Some factors that may be considered during the conversion process are the type of linear accelerator of the system, whether the couch or patient support is movable, whether a ring-type gantry or a C-arm is utilized, how a tumor is defined, and how dose is determined.

The software program 86 also includes a plan comparison module 150 illustrated in FIG. 5. The plan comparison module 150 is operable to compare treatment plans and assist the patient 14 in comparing and shopping for radiation therapy treatment. The patient 14 may elect to have pre-treatment (or mid-treatment or even post-treatment) data sent to a set of medical centers interested in generating potential treatment plans. The plan comparison module 150 can receive and transmit the patient data to a plurality of facilities for plan generation. The plan comparison module 150 can receive the generated plans and compare the different plans, the locations where treatment will be administered, treatment quality, side-effects, personnel on site, and other parameters and make a recommendation to the patient 14 based on certain requirements set forth by the patient 14. The patient 14 can then enlist in treatment at a preferred medical center. A planning center does not need to perform the treatment itself, as another option is for a remote planning center to export the plan to a local center where the treatment can be delivered.

Alternatively, the patient 14 can have the treatment plan evaluated by a consulting service to recommend a course of treatment. The remote service offered through the plan comparison module 150 can also be utilized during or after treatment for the patient 14 to receive feedback as to whether treatment adjustments are desired, and to evaluate if monitored changes in tumor, RAR, or side-effects are consistent with any doses prescribed or received.

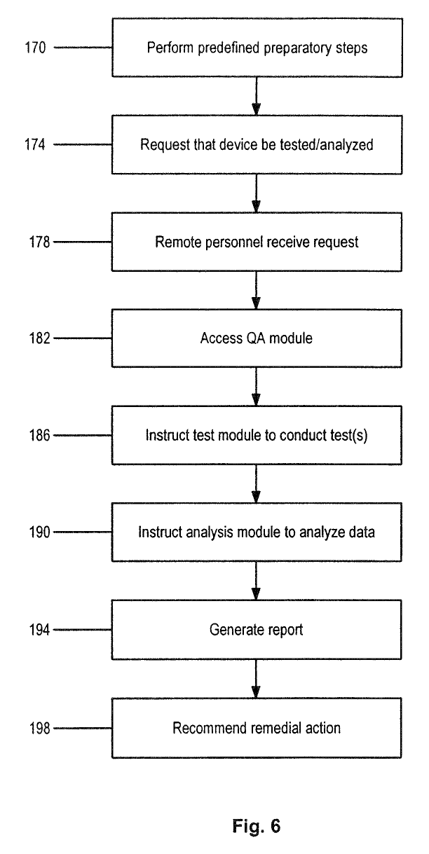

FIG. 6 illustrates a flow chart of a method of configuring a radiation therapy treatment device 18 from a remote location according to one embodiment of the invention. Local personnel perform (at 170) a set of predefined preparatory steps of the device 18, such as setting up equipment. Local personnel request (at 174) via the quality assurance module 98 that the device 18 be tested or analyzed for proper operation. Remote personnel receive (at 178) the request and access (at 182) the quality assurance module 98 via the remote computer 78 and the network 82. Remote personnel instruct (at 186) the test module 102 to conduct a particular test on the device 18 (e.g., conduct a test on operation of the gantry or the couch). After completion of the test, the remote personnel instruct (at 190) the analysis module 106 to evaluate the test results. The analysis module 106 generates (at 194) a report of the test results and transmits the report to the remote computer 78. The remote personnel recommend (at 198) remedial action if necessary. The analysis module 106 can automatically recommend changes to the device 18.

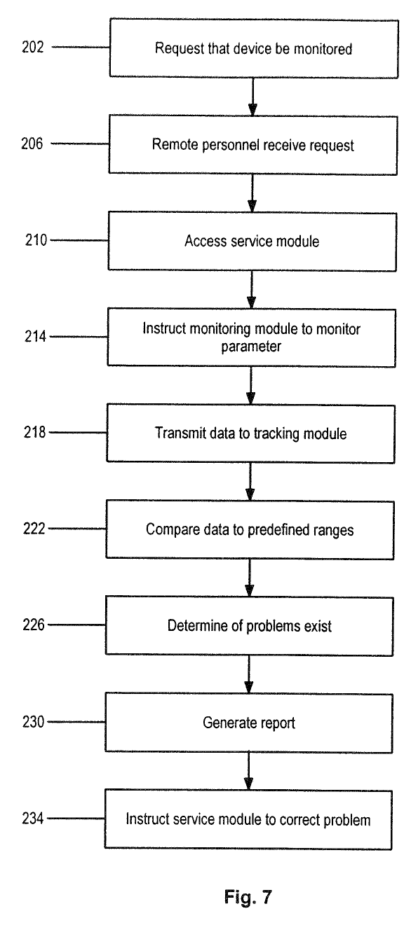

FIG. 7 illustrates a flow chart of a method of monitoring operation of a radiation therapy treatment device 18 from a remote location according to one embodiment of the invention. Local personnel request (at 202) via the service module 114 that the device 18 be monitored during operation or that environmental factors be evaluated or that external components be monitored. Remote personnel receive (at 206) the request and access (at 210) the service module 114 via the remote computer 78 and the network 82. Remote personnel instruct (at 214) the monitoring module 118 to monitor a parameter of the device 18, such as water flow, internal temperature, internal pressure, and the like or to monitor environmental factors, such as temperature, humidity, and air pressure or to monitor external components. The monitoring module 114 transmits (at 218) the acquired data to the tracking module 122. The tracking module 122 compares (at 222) the data to historical data or predefined ranges to determine (at 226) if device component problems or environmental problems or external component problems exist. The tracking module 122 generates (at 230) a report of the results and transmits the report to the remote computer 78. The remote computer 78 can access the tracking module 122 to retrieve the results. Based on the results, the remote personnel instruct (at 234) the service module 114 to correct the problem. The service module 114 can automatically correct the problem rather than wait for the report.

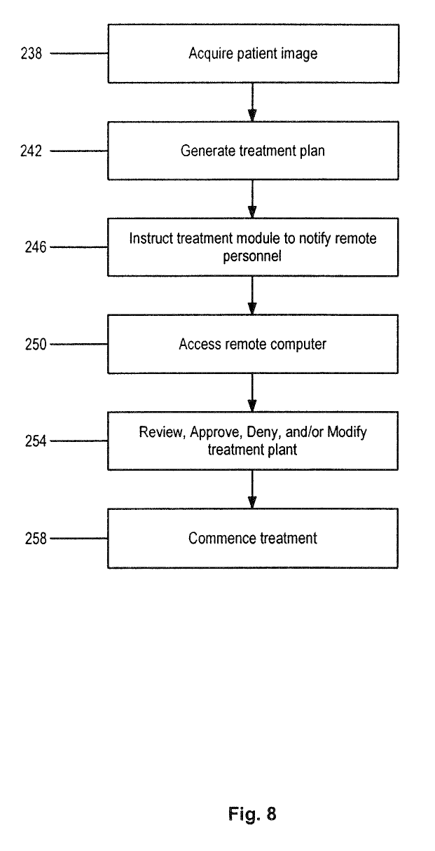

FIG. 8 illustrates a flow chart of a method of remotely reviewing a radiation therapy treatment plan for a patient according to one embodiment of the invention. Local personnel acquire (at 238) an image of the patient 14 and begin to generate (at 242) a treatment plan for the patient. The local personnel instruct (at 246) the treatment module 126 to notify remote personnel that a treatment plan has been generated. The remote personnel access (at 250) a computer 78 at a location different from the local personnel, and review, approve, modify, and/or deny (at 254) the treatment plan. Remote personnel can also view, edit, and/or approve patient plan optimization; view, edit, and/or approve patient contours; view, edit, and/or approve patient registration, and registration histories for a patient 14; view, edit, and/or approve adaptive therapy. If the remote personnel approve the treatment plan, the local personnel commence (at 258) treatment.

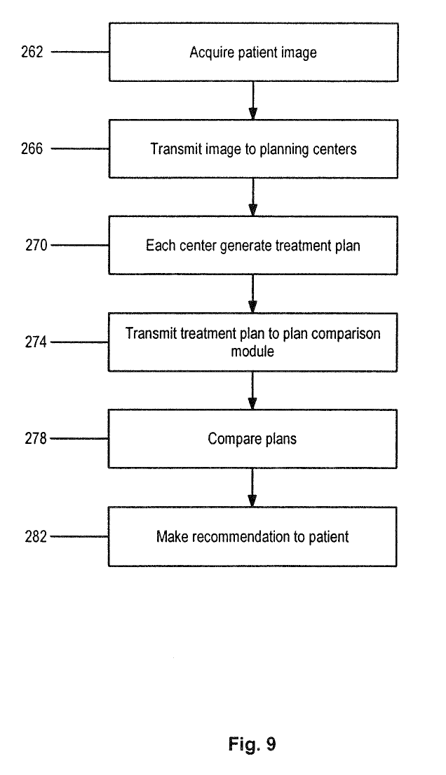

FIG. 9 illustrates a flow chart of a method of selecting a location for radiation therapy treatment according to one embodiment of the invention. Local personnel acquire (at 262) a patient profile (e.g., information or data relating to the patient) and transmit (at 266) the profile to a plurality of treatment planning locations. Each location generates (at 270) a treatment plan for the patient 14 based on the patient profile. Each location transmits (at 274) the treatment plan to the plan comparison module 150. The plan comparison module 150 compares (at 278) the plurality of plans to make (at 282) a recommendation to the patient 14 of where to receive treatment.

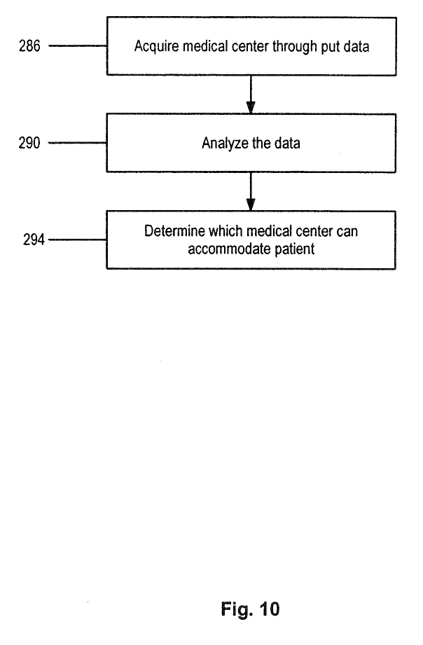

FIG. 10 illustrates a flow chart of a method of scheduling radiation therapy treatment for a patient 14 at a medical center according to one embodiment of the invention. A medical center data module 142 acquires (at 286) throughput data, such as speed and workload, from a plurality of medical centers having a radiation therapy system 10. The medical center data module 142 analyzes (at 290) the throughput data and determines (at 294) which medical center can accommodate the patient 14 most efficiently. The medical center data module 142 can also determine a particular treatment unit to use. The medical center data module 142 can also take into consideration patient willingness to travel, preference for particular clinical personnel, and other patient related factors.

Thus, the invention provides, among other things, new and useful systems and methods of remotely accessing a radiation therapy system. Various features and advantages of the invention are set forth in the following claims.

1. A method of remotely directing radiation therapy treatment of a patient, the radiation therapy treatment being delivered by a radiation therapy system, the method comprising:
remotely accessing patient information using a remote computer distinct from the radiation therapy system;
processing at least a portion of the patient information;
providing at least a portion of the processed information to an on-site person at the radiation therapy system;
wherein at least a portion of the treatment has been delivered to the patient; and
wherein the patient information is derived from the delivered portion.

2. A method as set forth in claim 1 and further comprising notifying a person at a location distinct from the radiation therapy system when a decision point has been reach during the treatment.

3. A method as set forth in claim 2 wherein the method further comprises determining the occurrence of the decision point at a fixed point in the treatment.

4. A method as set forth in claim 2 wherein the decision point occurs based on predefined actions.

5. A method as set forth in claim 2 wherein the notifying a person is via the remote computer.

6. A method as set forth in claim 5 wherein the remote computer comprises a hand-held device.

7. A method as set forth in claim 1 wherein the patient information is accessed at a second remote computer distinct from the radiation therapy system.

8. A method as set forth in claim 1 wherein the patient information is accessed at the radiation therapy system.

9. A method as set forth in claim 1 and further comprising approving or altering a course of treatment based at least in part on the processed information.

10. A method as set forth in claim 9 wherein the approving or altering the course of treatment includes approving or altering a radiation therapy treatment plan.

11. A method as set forth in claim 10 and further comprising performing the radiation therapy treatment plan in response to the approving or altering the radiation therapy treatment plan.

12. A method as set forth in claim 1 and further comprising generating a radiation therapy treatment plan for the patient based at least in part on the processed information.

13. A method as set forth in claim 12 wherein the providing act includes providing the radiation therapy treatment plan to the on-site person.

14. A method as set forth in claim 12 wherein the generating a radiation therapy treatment plan occurs at the radiation therapy system.

15. A method as set forth in claim 1 wherein the providing act includes delivering at least a portion of the processed information to the on-site person.

16. A method as set forth in claim 1 wherein the providing act includes making available at least a portion of the processed information to the on-site person.

17. A method as set forth in claim 1 wherein at least a portion of the provided information includes original, remotely-accessed patient information.

18. A method as set forth in claim 1 wherein the processing act includes generating a radiation therapy treatment plan, and wherein the providing act includes providing the radiation therapy treatment plan to the on-site person.

19. A method as set forth in claim 18 and further comprising providing the radiation therapy treatment plan to a person at a location distinct from the radiation therapy system to have the person approve the radiation therapy treatment plan.

20. A method as set forth in claim 1 wherein the processing act includes making a decision by a person at a location distinct from the radiation therapy system regarding approval, modification, or creation of a treatment plan, and wherein the providing act includes providing the decision.

21. A method as set forth in claim 1 wherein the processing act includes making a decision by a person at a location distinct from the radiation therapy system regarding approval, modification, or creation of a patient setup, and wherein the providing act includes providing the decision.

22. A method as set forth in claim 1 wherein the processing act includes making a decision by a person at a location distinct from the radiation therapy system regarding approval, modification, or creation of a patient registration, and wherein the providing act includes providing the decision.

23. A method as set forth in claim 1 wherein the processing act includes making a decision by a person at a location distinct from the radiation therapy system regarding approval, modification, or creation of patient contours, and wherein the providing act includes providing the decision.

24. A method as set forth in claim 1 wherein the processing act includes making a decision by a person at a location distinct from the radiation therapy system regarding approval, modification, or creation of a patient quality assurance plan, and wherein the providing act includes providing the decision.

25. A method as set forth in claim 1 wherein the processing act includes making a decision by a person at a location distinct from the radiation therapy system regarding approval, modification, or creation of an adaptive therapy plan, and wherein the providing act includes providing the decision.

26. A method of remotely directing radiation therapy treatment of a patient, the radiation therapy treatment being delivered by a radiation therapy system, the method comprising:
remotely accessing patient information using a remote computer distinct from the radiation therapy system;
processing at least a portion of the patient information;
generating radiation therapy information based at least in part on the processed information; and
providing the radiation therapy information to an on-site person at the radiation therapy system;
wherein at least a portion of the treatment has been delivered to the patient; and
wherein the patient information is derived from the delivered portion.

27. A method as set forth in claim 26 and further comprising notifying a person at a location distinct from the radiation therapy system when a decision point has been reach during treatment.

28. A method as set forth in claim 27 wherein the method further comprises determining the occurrence of the decision point at a fixed point in the treatment.

29. A method as set forth in claim 27 wherein the decision point occurs based on predefined actions.

30. A method as set forth in claim 26 wherein the notifying a person is via the remote computer.

31. A method as set forth in claim 30 wherein the remote computer comprises a hand-held device.

32. A method as set forth in claim 26 wherein the patient information is accessed at a second remote computer distinct from the radiation therapy system.

33. A method as set forth in claim 26 wherein the patient information is accessed at the radiation therapy system.

34. A method as set forth in claim 26 and further comprising, prior to the providing the radiation therapy information, approving or altering a radiation therapy treatment plan.

35. A method as set forth in claim 34 and further comprising performing the radiation therapy treatment plan in response to the approving or altering the radiation therapy treatment plan.

36. A method as set forth in claim 26 wherein the providing act includes delivering at least a portion of the radiation therapy information to the on-site person.

37. A method as set forth in claim 26 wherein the providing act includes making available at least a portion of the radiation therapy information to the on-site person.

38. A method as set forth in claim 26 and further comprising providing the radiation therapy information to a person at a location distinct from the radiation therapy system to have the person approve a radiation therapy treatment plan.

39. A method as set forth in claim 26 wherein the radiation therapy information includes a radiation therapy treatment plan and further comprising making a decision by a person at a location distinct from the radiation therapy system regarding approval or modification of the radiation therapy treatment plan, and providing the decision to the on-site person.

40. A method as set forth in claim 26 wherein the processing act includes making a decision by a person at a location distinct from the radiation therapy system regarding approval, modification, or creation of a patient setup.

41. A method as set forth in claim 26 wherein the processing act includes making a decision by a person at a location distinct from the radiation therapy system regarding approval, modification, or creation of a patient registration.

42. A method as set forth in claim 26 wherein the processing act includes making a decision by a person at a location distinct from the radiation therapy system regarding approval, modification, or creation of patient contours.

43. A method as set forth in claim 26 wherein the processing act includes making a decision by a person at a location distinct from the radiation therapy system regarding approval, modification, or creation of an adaptive therapy plan.

44. A system for remotely directing radiation therapy treatment of a patient, the radiation therapy treatment being delivered by a radiation therapy system, the system comprising:
a computer processor distinct from the radiation therapy system; and
a software program stored in a computer readable medium distinct from the radiation therapy system and accessible by the computer processor, the software program being executable by the computer processor to access patient information, process at least a portion of the patient information, and provide at least a portion of the processed information to an on-site person at the radiation therapy system.

45. A system as set forth in claim 44 wherein the software program is further executable by the computer processor to notify a person at a location distinct from the radiation therapy system when a decision point has been reached.

46. A system as set forth in claim 44 wherein the accessed patient information is from a second computer processor distinct from the radiation therapy system.

47. A system as set forth in claim 44 wherein the accessed patient information is from the radiation therapy system.

48. A system as set forth in claim 44 wherein the software program is further executable by the computer processor to generate a radiation therapy treatment plan for the patient based at least in part on the processed information.

49. A system as set forth in claim 48 wherein the providing act includes providing the radiation therapy treatment plan to the on-site person.

50. A system as set forth in claim 48 wherein the generating a radiation therapy treatment plan occurs at the radiation therapy system.

51. A system as set forth in claim 44 wherein the software program is further executable by the computer processor to provide the radiation therapy treatment plan to a person at a location distinct from the radiation therapy system to have the person approve the radiation therapy treatment plan.

52. A system as set forth in claim 44 wherein the providing at least a portion of the processed information includes delivering at least a portion of the processed information to the on-site person.

53. A system as set forth in claim 44 wherein the providing at least a portion of the processed information includes making available at least a portion of the processed information to the on-site person.

54. A system as set forth in claim 44 wherein the processing at least a portion of the patient information includes requesting a decision by a person at a location distinct from the radiation therapy system to approve, modify, or create a treatment plan.

55. A system as set forth in claim 44 wherein the processing at least a portion of the patient information includes requesting a decision by a person at a location distinct from the radiation therapy system to approve, modify, or create a patient setup.

56. A system as set forth in claim 44 wherein the processing at least a portion of the patient information includes requesting a decision by a person at a location distinct from the radiation therapy system to approve, modify, or create a patient registration.

57. A system as set forth in claim 44 wherein the processing at least a portion of the patient information includes requesting a decision by a person at a location distinct from the radiation therapy system to approve, modify, or create patient contours.

58. A system as set forth in claim 44 wherein the processing at least a portion of the patient information includes requesting a decision by a person at a location distinct from the radiation therapy system to approve, modify, or create a patient quality assurance plan.

59. A system as set forth in claim 44 wherein the processing at least a portion of the patient information includes requesting a decision by a person at a location distinct from the radiation therapy system to approve, modify, or create an adaptive therapy plan.
